(19) United States
(12) Patent Application Publication
(10) Pub. No.: US 2007/0238940 A1
(43) Pub. Date: Oct. 11, 2007

(54) VIBRATOR PILL FOR GASTROINTESTINAL DISORDERS

(76) Inventor: Omar Amirana, Boston, MA (US)

Correspondence Address: VIERRA MAGEN MARCUS & DENIRO LLP, 575 MARKET STREET SUITE 2500, SAN FRANCISCO, CA, 94105, US

(21) Appl. No.: 11/561,964

(22) Filed: Nov. 21, 2006

Related U.S. Application Data
(60) Provisional application No. 60/739,177, filed on Nov. 23, 2005.

(51) Int. Cl. A61B 5/07; A61H 1/00

(52) U.S. Cl. 600/302; 601/46

(57) ABSTRACT

A vibrating pill is disclosed capable of stimulating the gastrointestinal tract to induce gastrointestinal contractions and peristalsis, as well as other functions within the gastrointestinal tract.

### CROSS-REFERENCE TO RELATED APPLICATION

The present application claims priority to provisional patent application Ser. No. 60/739,177, entitled, “Vibrator Pill,” filed Nov. 23, 2005, which application is incorporated by reference herein in its entirety.

### BACKGROUND OF THE INVENTION

1. Field of the Invention

Embodiments of the present invention relate to a vibrating pill capable of stimulating the gastrointestinal tract to induce gastrointestinal contractions and peristalsis, as well as other functions within the gastrointestinal tract.

2. Description of the Related Art

Gastrointestinal motility disorders are a major problem. Ileus, or paralysis of the gastrointestinal tract, is a significant and life threatening problem. Often postoperative ileus prevents patients from leaving the hospital and can result in surgical intervention. In many cases of dysmotility, a gastrointestinal stimulant is required and desired. Worldwide, many people drink coffee or consume additional fiber in their diets to maintain regular bowel movements. Gastroparesis, ileus and constipation are all significant gastrointestinal motility disorders.

In the gastrointestinal arena, both pharmaceutical and device stimulators are being used to help control bowel function with applications in obesity, ileus, gastroparesis and dysmotility syndromes. To the best of my knowledge, no device exists to date which is ingestible, capable of being remotely activated and programmed which also produces a therapeutic mechanic effect from within the lumen of the bowel. This invention is a remotely activated and programmed, mechanically vibrating pill capable of stimulating the gastrointestinal tract to induce gastrointestinal contractions and peristalsis. One particular application of this invention is to treat ileus and to relieve constipation.

### SUMMARY OF THE INVENTION

One embodiment relates to a vibrating device (“Pill”) that can be ingested to stimulate the gastrointestinal (GI) tract using an internal vibrating mechanism that is capable of being varied in intensity, duration, pulse, and strength and can be activated, deactivated, or altered remotely.

Pill that also has an internal magnet that is maneuvered and positioned magnetically.

Pill that also has an internal camera that is capable or capturing images and video data.

Pill that is maneuvered and positioned magnetically and is capable or capturing images and video data that can be wirelessly transmitted from the body and processed and evaluated remotely.

Pill with magnet and electrodes that is maneuvered and positioned magnetically and can mechanically and electrically stimulate tissue.

Pill with camera, magnet, vibrating capability, and electrodes that is maneuvered and positioned magnetically and can mechanically and electrically stimulate tissue while capturing and transmitting image data.

Pill with camera, magnet and electrodes that is maneuvered and positioned magnetically and can mechanically and electrically stimulate tissue while capturing and transmitting image data that also stores and selectively releases medicine.

### DESCRIPTION OF THE DRAWINGS

FIG. 1 is an illustration of a programmable, controllable, maneuverable ingestible device.

FIG. 2 is a system for controlling an ingestible device.

### DETAILED DESCRIPTION

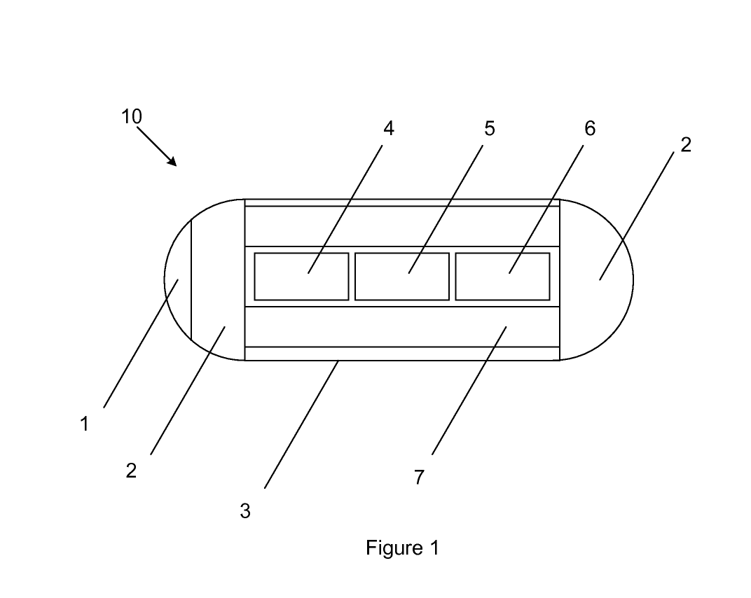

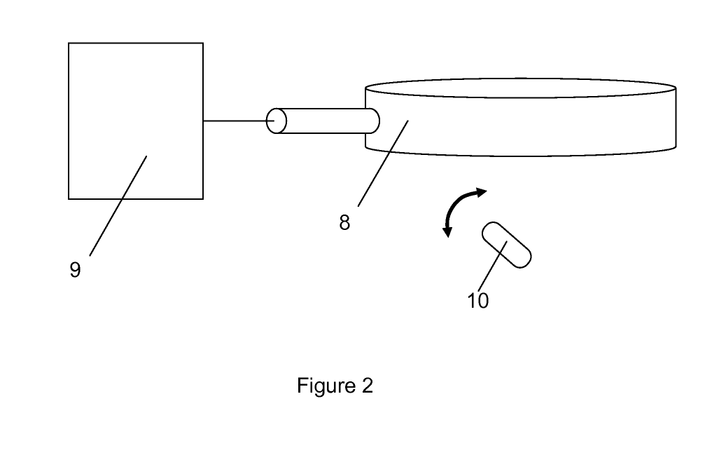

Embodiments will now be described with reference to FIGS. 1 through 2, which relate to a semiconductor device having tightly offset semiconductor die. It is understood that the present invention may be embodied in many different forms and should not be construed as being limited to the embodiments set forth herein. Rather, these embodiments are provided so that this disclosure will be thorough and complete and will fully convey the invention to those skilled in the art. Indeed, the invention is intended to cover alternatives, modifications and equivalents of these embodiments, which are included within the scope and spirit of the invention as defined by the appended claims. Furthermore, in the following detailed description of the present invention, numerous specific details are set forth in order to provide a thorough understanding of the present invention. However, it will be clear to those of ordinary skill in the art that the present invention may be practiced without such specific details.

An embodiment of the invention shown in FIG. 1 is a programmable, controllable, maneuverable ingestible device (10). The device (10) is capable of safely being swallowed, whether absorbed or not or excreted or not. The device (10), hereafter referred to as a “Pill” contains one or multiple vibrating element(s) (7) capable of being remotely activated, deactivated and programmed to increase, decrease, terminate or initiate their mechanical action. The vibrating element in the device (10) enables the patient or other to remotely control the intensity of the vibrating action of the device as it traverses the patient's gastrointestinal system. While use of vibrating elements within an ingestible pill is thought to be novel, vibrating elements for such use are known in the art. The vibrating element may run off a battery (5).

In addition to the vibrating element used to mechanically stimulate the gastrointestinal tract, it also contain may contain some or all of the following characteristics: a non conducting physiologically inert outer casing (3), a camera (4) capable of capturing image (static and/or video) data through an aperture (1), circuitry capable of storing, transmitting and receiving data such as image, electrical and physiologic data (6), electrodes (2) and circuitry (6) capable of sensing and producing electrical signals, whether direct current or alternating current, whether intermittent or continuous, whether or not capable of stimulating the gastrointestinal tract or simply for diagnostic purposes such as localization, circuitry enabling remote activation and deactivation as well as programmability, radioactive elements for various uses such as cancer therapy, drug delivery capability to specifically release medication or compounds into the gastrointestinal tract at a given location, Pill-to-Pill as well as Pill-to-System wireless communication for timed or gated diagnostic or therapeutic actions using multiple Pills such as, but not limited to, physiologic data capture including images, or for drug release or for electrical activity including gastrointestinal stimulation. In embodiments, medicine may be stored within the device (10) and released into the lumen of the bowel over time as is known in the art. For example, the portion of the device (10) storing the medicine may include a permeable or semi-permeable membrane allowing the medicine to diffuse into the lumen over time.

As shown in FIG. 2, the system also includes a communication and programming system comprising of control box (9) to control and vary various input settings, a communication device such as a handheld wand (8) which communicates remotely with the Pill (10) in order to activate, deactivate, program or vary its settings.

Embodiments of this invention relate to an ingestible mechanical stimulation device that would treat various gastrointestinal conditions such as gastrointestinal motility disorders including but not limited to ileus, gastroparesis and constipation which could also include various additional diagnostic and therapeutic features.

The foregoing detailed description of the invention has been presented for purposes of illustration and description. It is not intended to be exhaustive or to limit the invention to the precise form disclosed. Many modifications and variations are possible in light of the above teaching. The described embodiments were chosen in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

I claim:

1. A system comprising:
an ingestible device with vibrating or mechanically stimulating properties of remotely programmable intensity, wherein the ingestible device can be remotely and selectively and externally activated, programmed and deactivated within the gastrointestinal tract, to produce physiologic effects, including one or more of:
a. peristalis;
b. gastrointestinal motility;
c. bowel movements or defecation.

2. An device ingestible by a person, comprising:
a receiver for receiving a signal originating outside of the person; and
an element capable of vibrating to cause the gastrointestinal tract or bowel to contract, and the element capable of being safely passed through the lumen of the gastrointestinal tract.

3. A system according to claim 1, wherein the ingestible device contains a magnetic element for remotely and selectively maneuvering and positioning the ingestible device into one location within the gastrointestinal tract using an external system.

4. A system according to claim 3, wherein the external system uses an external electromagnetic field.

5. A system according to claim 1, wherein the ingestible device includes one or more components to capture and store or capture and wirelessly transmit image data of the gastrointestinal tract.

6. A system according to claim 5, wherein the transmitted data is captured and processed by an external data processing system for evaluation in real time during or after the procedure.

7. A system according to claim 1, wherein the ingestible device has the ability to store and selectively release medication or compounds into gastrointestinal tract.

8. A system according to claim 1, in which the ingestible device has the ability to store and selectively release medication or compounds into gastrointestinal tract.

9. A system according to claim 1, wherein the ingestible device has one or a plurality of electrodes capable of sensing and storing or sensing and transmitting an electrical signal or signals.

10. A system according to claim 10, wherein the signals from the one or a plurality of electrodes are capable of stimulating the gastrointestinal tract.

11. A system according to claim 1, wherein the ingestible device has one or a plurality of electrodes capable of sensing and producing an electrical signal or signals where the electrical signal is a current signal either of continuous or intermittent duration such as a pulsed signal which is capable of being sensed by sensors placed on the skin, including but not limited to:
a. an embodiment where the signal is sensed on the skin at multiple locations simultaneously;
b. an embodiment where the signal is sensed on the skin at multiple locations simultaneously to calculate the three dimensional position of the ingestible device at a given point in time and space;
c. an embodiment where the signal(s) are sensed on the skin at multiple locations simultaneously to calculate the three dimensional position of the ingestible device at a given point in time and space, and this is performed numerous times in order to reconstruct a three dimensional image of the lumen of the gastrointestinal tract;
d. an embodiment where the signal(s) are sensed on the skin at multiple locations simultaneously to calculate the three dimensional position of the ingestible device at a given point in time and space, and this is performed a plurality of times in order to reconstruct a three dimensional image of the lumen of the gastrointestinal tract and also to simultaneously superimpose the gastrointestinal tract's electrical activity as sensed by the Pill onto the three dimensional reconstruction.

12. A system according to claim 3, wherein the ingestible device has one or a plurality of electrodes capable of sensing and producing an electrical signal or signals where the electrical signal is a current signal either of continuous or intermittent duration such as a pulsed signal which is capable of being sensed by sensors placed on the skin, the one or plurality of electrodes including:
a. an embodiment where the signal is sensed on the skin at multiple locations simultaneously;
b. an embodiment where the signal is sensed on the skin at multiple locations simultaneously to calculate the three dimensional position of the ingestible device at a given point in time and space;
c. an embodiment where the signal(s) are sensed on the skin at multiple locations simultaneously to calculate the three dimensional position of the ingestible device at a given point in time and space, and this is performed numerous times in order to reconstruct a three dimensional image of the lumen of the gastrointestinal tract;
d. an embodiment where the signal(s) are sensed on the skin at multiple locations simultaneously to calculate the three dimensional position of the ingestible device at a given point in time and space, and this is performed numerous times in order to reconstruct a three dimensional image of the lumen of the gastrointestinal tract and also to simultaneously superimpose the gastrointestinal tract's electrical activity onto the three dimensional reconstruction or to superimpose the gastrointestinal tract's visual appearance onto the three dimensional reconstruction.

13. A system according to claim 1, wherein the ingestible device has one or a plurality of sensors capable of sensing pressure and producing an electrical signal or signals with integrated feedback control such that the system can alter or terminate mechanical stimulation and or signal production based on the information sensed.

14. A system according to claim 1, wherein the ingestible device includes an element emitting ionizing radiation, and including one or more of:
a. various radioactive elements of various strengths and rates of decay to create a therapeutic effect;
b. various radioactive elements of various strengths and rates of decay to create a therapeutic effect, specifically gastrointestinal cancers including but not limited to colon cancer
c. various radioactive elements of various strengths and rates of decay to create a therapeutic effect, specifically non-gastrointestinal cancers including but not limited to pancreatic cancer, liver cancer, or cancer of the prostate.

15. A system according to claim 14, wherein the element emitting ionizing radiation is a radioactive pellet or seed.

16. A system according to claim 1, wherein the device is coated with a pleasant tasting, dissolvable or degradable coating.

17. A system according to claim 1, wherein the ingestible device is of dimensions between than 0.1 millimeter and 20 millimeters in length or width or depth.

18. A system according to claim 1, wherein the ingestible device can be activated, programmed and deactivated either before or after being ingested using a noninvasive system from outside the skin where each device has its own communication or telemetry frequency such that one device does not interfere with another device if multiple devices are ingested in the same period.

19. A system and device according to claim 1, wherein a plurality of ingestible devices are used concomitantly and the devices are capable of communicating with a control station independently as well as with one another to effect a therapeutic effect, including one or more of:
a. timed or gated mechanical stimulation, such as to affect peristalsis;
b. timed or gated data capture and transmission;
c. timed or gated mechanical and electrical stimulation;
d. timed or gated medication or compound release.
